(19) United States
(12) Patent Application Publication
(10) Pub. No.: US 2010/0266555 A1
(43) Pub. Date: Oct. 21, 2010

(54) MICRO RNAS AS MARKERS OF THE FUNCTIONAL STATE OF A DENDRITIC CELL

(75) Inventors: Simon Skjøde Jensen, Bronshoj (DK); Kim Holmstrøm, Ballerup (DK); Ayako Wakatsuki Pedersen, Frederiksberg (DK); Mai-Britt Zocca, Copenhagen (DK)

Correspondence Address: THE NATH LAW GROUP, 112 South West Street, Alexandria, VA, 22314, US

(73) Assignees: DANDRIT BIOTECH A/S, Copenhagen Ø (DK); BIONEER A/S, Hørsholm (DK)

(21) Appl. No.: 12/741,801

(22) PCT Filed: Nov. 14, 2008
(86) PCT No.: PCT/DK2008/000406

§ 371 Date: May 6, 2010

(30) Foreign Application Priority Data
Nov. 14, 2007 (DK) PCT/DK2007/000501

(51) Int. Cl. A61K 35/28; C12Q 1/68

(52) U.S. Cl. 424/93.7; 435/6

(57) ABSTRACT

The present invention relates to the use of specified micro RNAs as markers of the functional state of a dendritic cell. In one aspect the invention relates to a method for producing a quality-controlled therapeutic composition comprising dendritic cells. In another aspect the invention relates to a method of in vitro screening of immunomodulatory compounds.

### TECHNICAL FIELD

The present invention relates to the use of one or more micro RNAs comprising specific sequences as markers of the functional state of a dendritic cell.

The invention further relates to a method of producing a quality-controlled composition comprising dendritic cells, wherein the functional state of the dendritic cells in the composition is monitored.

The invention further relates to a method for in vitro screening for compounds having immunomodulatory effect.

### BACKGROUND

Dendritic cells (DC) are bone marrow-derived cells that function as professional antigen presenting cells (APC) of the immune system. DC have distinct states of cell development and activation and have the potential to induce both immunity and tolerance. For the purpose of the present invention, the functional states of dendritic cells are defined as a) immature, b) mature and c) tolerogenic. “Mature” dendritic cells are also often referred to as “immunogenic” dendritic cells.

In an inflammatory environment DC in the periphery (immature DC) become activated, which leads to phenotypic and functional changes whereby the DC enter a new functional stage. When differentiating into immunogenic dendritic cells, DC will show a diminished antigen uptake and processing capabilities and become equipped with enhanced antigen presentation accompanied by expression of adhesion and co-stimulatory molecules, both of which are required for induction of T-cell activation. After migrating to lymphoid organs, DC (now mature immunogenic DC) present the antigen to T-cells in the presence of co-stimulatory signals and the T cell response is initiated. DC are unique in that they are the only APC that are capable of triggering not only memory responses, but also naïve T cells.

Under steady-state conditions, i.e. in the absence of inflammation or infection, DC can still migrate from the periphery to the secondary lymphoid organs, where they encounter T cells and differentiate, but without triggering T cell activation. Such differentiated DC era called differentiated tolerogenic DC. These DC contribute to the maintenance of peripheral tolerance (therefore these DC are often termed “tolerogenic DC”).

The crucial role of DC as a powerful inducer of immune response renders the DC one of the most promising platforms for active immunotherapy in various infections and in cancer.

In clinical trials, employing DC as an immunotherapeutic vaccine, DC are enriched or differentiated from their precursors due to their low frequency in the peripheral blood. In most trials, this is done with peripheral blood monocytes, which can be obtained from patients' blood or leukopheresis products. The monocytes are cultured in the presence of GM-CSF and IL-4, which leads to a differentiation to immature DC. Functionally immunogenic DC can be produced by numerous methods, but most vaccination trials have used DC differentiated with a combination of TNFα, IL-1β and IL-6 (the active substances of the so-called monocyte-conditioned medium) and PGE₂.

One major obstacle with the currently ongoing immunotherapy trials using ex vivo cultured DC is that it is difficult to predict the function of DC once they are administered in vivo. (For example, to elicit an anti-tumour response in cancer patients, the DC have to be able to polarise a Th1-T-cell response, dominated by IFNγ production). Therefore, it has been suggested that the optimal DC suited for cancer vaccination should have a mature phenotype, indicated by the expression of CD83 and T cell co-stimulatory molecules such as CD40, CD80, CD86, retained migratory abilities (CCR7 expression), and preferably secretes Th1-promoting cytokines such as IL-12p70 and no immuno-suppressive cytokines such as IL-10 (Figdor et al 2004 Nat Med (10) 475-80). Given the dynamic nature of DC, it is essential that one would carry out quality controls of the DC used for vaccination.

Currently, the most common method for monitoring the function of DC before vaccination is by examining the expression of cell surface markers, Whilst this gives an idea of the extent of the functional state of a DC population, it is far from a complete and certain picture. Thus, any additional tool that allows a broader phenotypic characterisation of DC would be very beneficial.

Thus, it is an object of the present invention to provide an alternative or improved method for monitoring the functional state of a dendritic cell.

Micro RNAs (miRNAs) are small non-coding RNA molecules present in plants, animals and humans. The discovery of these 19-25 nucleotide long miRNA molecules have introduced a new level of post-transcriptional control of gene expression through the mechanism of RNA-interference (RNAi) first documented by Fire et al. in 1998 (Nature, vol. 391:806).

Micro RNAs are genetically encoded in the human genome and are expressed initially as a primary transcript (pri-miRNA) in the nucleus of the cell. Micro RNAs can both be expressed as mono-specific or poly-cistronic primary transcripts, which undergo processing into a precursor (pre-miRNA) prior to being exported to the cytoplasm of the cell, wherein the final processing into the mature miRNA occurs.

The mature miRNA does not function as naked RNA, but instead as components of ribonucleoprotein complexes (RNFs), which promote the translational arrest of the target messenger RNA (mRNA) by binding typically in the 3′ untranslated region (3′-UTR). The mature miRNA binds to the target, however, not necessarily in 100% complementary fashion, and a favorable conformational equilibrium between the mRNA target and the RNP complex as such is most likely also important for the specific interaction. Single miRNAs are believed to control several genes in a complex regulatory network, which is only beginning to be understood. Today, there is evidence for multiple modes of miRNA-mediated regulation, including translational inhibition, increased mRNA deadenylation and/or degradation and mRNA sequestration.

A total of 1000 miRNAs have been predicted in the human genome based on sequence information and it has been suggested that approximately 30% of the total number of the 22,000 genes present in the genome are under control of miRNAs.

In the latest version 10.0 of miRBase (release date August 2007), Sanger Institute, 722 different validated human miRNAs have been published. Interestingly, many miRNAs are highly similar across organisms indicating an evolutionarily important role of miRNA and they presumably control very fundamental biological processes in higher cells. Animal examples of documented miRNA functions include regulation of signaling pathways, apoptosis, metabolism, cardiogenesis and brain development, and currently the elucidation of the biological significance of miRNAs is under intense investigation in the scientific community.

The use of high-through-put technologies like microarray and real-time PCR analyses have contributed extensively to the knowledge of the presence or absence of miRNAs in different types of diseased or normal cells and tissues. Micro RNA expression levels and profiles vary from tissue to tissue, between normal and malignant cells, and during cell differentiation and development. Hence, the potential use of miRNAs as a novel type of biomarker has been proposed.

Particularly in cancer, the promise of using miRNAs as new alternative biomarkers have been demonstrated, and in the pioneering work of Lu et al. (Nature (2005), vol. 435:834) it was shown that miRNA profiling compared to traditional mRNA profiling provided a much more precise molecular taxonomy and classification of tumor samples of different origin.

Micro RNA profiles or even specific miRNAs can be associated with specific phenotypic characteristics of cells to distinguish for example between normal and tumor cells, stem cells and differentiated cells.

The involvement of specific miRNAs in immune responses has been shown in several publications. A. Rodriguez at al. (Science, vol. 316, 608-611, 2007) showed that mice deficient for the miRNA-155-gene (miRNA-155^{KO-mice}) are immunodeficient in several ways. The mice showed decreased resistance against a salmonella infection after a vaccination trial showing that the mice are defective in adaptive immunity. Dendritic cells from miRNA-155^{KO-mice} showed reduced ability to stimulate antigen specific proliferation of OVA TCR transgenic T-cells, with reduced T-cell secretion of IL-2, a typical Th1-produced cytokine.

Development of naive T-cells towards the Th1-differentiation was not affected, whereas Th2-differentiation was promoted. Taken together, Rodriguez et al. show that lack of the miRNA-155-gene causes a deficiency in normal immune function in mice. Rodrigues at al. do not show that miRNA-155 is upregulated in dendritic cells having a mature phenotype.

Taganov at al. (PNAS, vol 103, no. 33, 12481-12486, 2006) show that two human monocytic/macrophage like cell lines THP1 and HL-60 respond to different TLR-agonists by induction of miRNA-155, miRNA-132 and miRNA-146a/b. In particular, miRNA-146 is induced by Pam3CSK4 (TLR2), Peptidoglycan (TLR2), Poly I:C (TLR3), LPS (TLR4), Flagellin (TLR5), R848 (TLR7), CpG (TLR9) and cytokines TNFα, IL1β and CD40 ligand. Taganov et al. do not study miRNA-profiles in dendritic cells. Although Taganov et al. refer to publications in which expression of miRNA-155 is increased in activated macrophages and dendritic cells (Taganov at al. and Stetson et al.), the authors do not show or suggest that miRNA-155 is upregulated in dendritic cells having a mature phenotype.

O'Connell R. M. at al. (PNAS, vol 104, no. 5, 1604-1609, 2007) showed that miRNA-155 was induced in macrophages derived from mouse bone marrow cells, treated with the cytokines TNFα and IFNβ or the Toll like receptor (TLR) agonists Poly I:C (TLR3), LPS (TLR4), CpG (TLR9) and Pam3CSK4 (TLR2). This shows that miRNA-155 is involved in recognition of pathogen-associated molecular patterns activating the innate immune response in mice. Although macrophages are in family with dendritic cells, both being antigen presenting cells, dendritic cells are believed to be the major professional antigen presenting cell with ability to induce strong T and B-cell activation. The authors do not show that miRNA-155 is upregulated in dendritic cells having a mature phenotype.

One object of the invention was to identify miRNA sequences that are differentially expressed in dendritic cells of different phenotypes which are suitable for use to distinguish between immature, tolerogenic and immunogenic dendritic cells.

### DISCLOSURE OF THE INVENTION

Definitions

In the context of the present application, the definitions given below are applicable.

“Micro RNA” or “miRNA” means endogeneous non-coding oligoribonucleotides (RNA) of approximately 22 nucleotides (range 19-25) involved in posttranscriptional gene repression and translational arrest caused, typically, by binding to the 3′-untranslated regions (3′-UTR) of specific messenger RNAs (mRNAs).

“Micro RNA expression profile” or “expression profile” means a quantitative (absolute or relative) representation of the expression level of one or more micro RNAs present at a certain time in a certain sample, typically determined by microarray technology, employing arrays comprising specific complementary probe sequences to the micro RNAs in question, or by other methods like multiple parallel micro RNA real-time PCR based detection technology and fluorescent bead-based expression profiling (e.g. Luminex).

“Standard” refers to a standard micro RNA expression profile, which may be used as a basis for comparison used to calculate and determine the differential expression or the absolute amount (normalised to total-RNA content) of one or more micro RNAs at a certain time in a certain sample. The standard may be a relative standard, e.g. generated from an extract of total RNA from a reference set of cells (e.g. immature DC) used for relative quantification of differential expression of micro RNAs, or it may be an absolute or universal standard, e.g. generated from an extract of total-RNA of DC in a validated functional state used to establish a standard curve for absolute (normalised to total-RNA amounts) quantification, e.g. by employing real-time PCR.

“Dendritic cell” means an antigen presenting cell apart from macrophages and B-cells that possess the ability to 1) phagocytose foreign particles in a certain state, 2) develop dendrities in the mature state, 3) regulate the adaptive immune system through for example induction of Th1-cells or regulatory T-cells, 4) respond to pattern-associated molecular patterns (PAMP) through the innate immune system, e.g. toll receptor activation, and are capable of 5) cross-presentation of an antigen.

“Immature dendritic cell” means a cell in a state of differentiation from for example a monocyte that has been treated in a specific manner, typically with CM-CSF and IL4. Immature dendritic cells are characterised by high endocytic activity and low T-cell activation potential and respond to danger signals and/or combinations of cytokines or chemokines in its sourroundings through interaction with specific receptors. Immature dendritic cells phagocytose pathogens and degrade its proteins into small pieces and upon maturation present those fragments at their cell surface using MHC molecules. Once the immature dendritic cells have come into contact with a pathogen or cytokine or chemokines, they become activated into mature dendritic cells. Immature dendritic cells typically show low levels of surface receptors HLA-DR, CD40, CD80, CD83, CD86 and CCR7. Immature dendritic cells furthermore show high levels of surface receptor CD1a and low levels of the monocyte marker CD14.

“Immunogenic dendritic cell” or “mature dendritic cell” or “immunogenic differentiated dendritic cell” all mean a dendritic cell that is derived from an immature dendritic cell exposed to a differentiation stimulus, which can be either of microbial or pathogen origin, combinations of cytokines and/or chemokines, whereby the dendritic cell acquires the ability of inducing an immune response. An immunogenic dendritic cell has low endocytic activity, but high ability to regulate T-cell function, e.g. activation of Th1-cells. Mature dendritic cells typically show high expression levels of surface receptors HLA-DR, CD40, CD80, CD83 and CD86.

“Tolerogenic dendritic cell” means a dendritic cell that is derived from an immature dendritic cell exposed to a differentiation stimulus, which can be a combination of cytokines, hormones, vitamins and other biological agents whereby the dendritic cell acquires the ability of inducing tolerance. A tolerogenic dendritic cell has low ability to activate effector T cells but high ability to induce and activate regulatory T cells.

“Immunogenic” or “mature” means “capable of inducing an adaptive immunological response”.

“Tolerogenic” means “capable of silencing or down-modulating an adaptive immunological response”.

“Immunomodulating” means “capable of modifying an innate or an adaptive immunological response”.

“Autoimmune disease” means a pathological condition In which the adaptive immune system is directed against self antigens in a destructive manner.

“Vaccine” means an immunogenic or antigenic substance, which after introduction into an animal or human induces an immune response directed against the vaccine antigen(s) and thereby protects an individual for instance against infectious or allergic diseases or cancer diseases.

“Reference sequence” means a sequence shown in table 1 or table 2.

“Essentially homologous” means having a sequence deviating from the reference sequence at only 4 nucleobases or less, preferably at only 3 nucleobases or less, even more preferably at only 2 nucleobases, or less and even more preferably deviating from the reference sequence at only one nucleobase. In terms of % identity this correspond to a level of identity of 80%, 85%, 90% or 95%, respectively, calculated using a miRNA sequence having a length of 20 bases.

In a preferred aspect “essentially homologous” means comprising a nucleic acid sequence which has at least 70% identity to the nucleic acid sequence of the reference sequence. More specifically, “essentially homologous” means comprising a nucleic acid sequence which has at least 75%, such as at least 76, 77, 78, 79, 80, 81, 82, 83, 84, 85, 86, 87, 88 or 89% identity to the reference sequence. Even more preferably, “essentially homologous” means comprising a nucleic acid sequence which has at least 90%, such as at least 91, 92, 93, 94, 95, 96, 97, 98, 99, 100% identity to the reference sequence.

In the most preferred meaning the term “essentially homologous” means comprising a sequence that does not deviate from the reference sequence.

In the most preferred meaning the term “essentially homologous” means having a sequence that does not deviate from the reference sequence. The reference sequences in terms of the present invention are the sequences according to the invention. In practice, there are examples of miRNAs that have been seen to “shift” or “change” sequence as the exact miRNAs are studied in more detail. When occurring, these shifts usually result in a deletion or an addition of 1-4 or even up to 7 bases in either of the ends (5′ or 3′) of the transcript. This does not reflect a real change in actual sequence, but merely the better understanding of the actual sequence and its maturation process. Thus, the sequences according to the invention may change insignificantly at the ends as future research on them progress.

“Functional state” means the physiological state of a dendritic cell being either immature, immunogenic or tolerogenic.

“Adjuvant” means a substance that enhances the immune response when administered with an antigen.

“Adaptive transfer” means transfer of cell-mediated immunity by, for example, transfer of lymphocytes.

The present invention is based on the finding that several micro RNA molecules are differentially expressed in immature versus immunogenic dendritic cells and in immature versus tolerogenic dendritic cells. The micro RNA expression profile of such differentially expressed micro RNA molecules can be used to monitor the functional state of dendritic cells.

Accordingly, in one aspect the invention relates to the use of one or more micro RNAs comprising a sequence that is essentially homologous to a sequence selected from the group consisting of SEQ IDs 1 to 45 as markers of the functional state of a dendritic cell.

More specifically the present inventors found that several micro RNA molecules are differentially expressed in immature versus immunogenic dendritic cells. The micro RNA expression profile of such differentially expressed micro RNA molecules can be used to distinguish between immature and immunogenic dendritic cells.

Accordingly, in one aspect the invention relates to the use of one or more micro RNAs comprising a sequence that is essentially homologous to a sequence selected from the group consisting of SEQ IDs 1 to 36 as markers to distinguish between immature and immunogenic dendrite cells.

Even more specifically the present inventors found that several micro RNA molecules are differentially expressed in immature versus tolerogenic dendritic cells. The micro RNA expression profile of such differentially expressed micro RNA molecules can be used to distinguish between immature and tolerogenic dendritic cells.

Accordingly, in one aspect the invention relates to the use of one or more micro RNAs comprising a sequence that is essentially homologous to a sequence selected from the group consisting of SEQ IDs 26 to 45 as markers to distinguish between immature and tolerogenic dendritic cells.

The differentially expressed micro RNAs and/or a micro RNA expression profile or one or more thereof may thus be used to monitor the state of therapeutic compositions comprising dendritic cells.

Accordingly, in one aspect the invention relates to a method of producing a quality-controlled composition comprising dendritic cells, wherein the functional state of the dendritic cells in the composition is or has been monitored, the method comprising the steps of
a) extracting nucleic acids from the dendritic cells from the composition,
b) monitoring the micro RNA expression profile of one or more micro RNAs comprising a sequence that is essentially homologous to a sequence selected from the group consisting of SEQ IDs 1 to 45 extracted in step a), and
c) comparing the micro RNA expression profile obtained in b) with a standard.

### DETAILED DESCRIPTION OF THE INVENTION

The present invention is based on the finding that several micro RNA molecules are differentially expressed in immature versus immunogenic dendritic cells and in immature versus tolerogenic dendritic cells. The micro RNA expression profile of such differentially expressed micro RNA molecules can be used to monitor the functional state of dendritic cells.

The micro RNA sequences which are differentially expressed are shown in Table 1 (showing micro RNA sequences differentially expressed in immature versus immunogenic dendritic cells) and Table 2 (showing micro RNA sequences differentially expressed in immature versus tolerogenic dendritic cells) below.

TABLE 1

| Seq-ID | miRBase name | Sequence 5′-3′ |
|---|---|---|
| 1 | hsa-miR-27a | UUCACAGUGGCUAAGUUCCGC |
| 2 | hsa-miR-23b | AUCACAUUGCCAGGGAUUACC |
| 3 | hsa-miR-27b | UUCACAGUGGCUAAGUUCUGC |
| 4 | hsa-miR-155 | UUAAUGCUAAUCGUGAUAGGGGU |
| 5 | hsa-miR-202 | AGAGGUAUAGGGCAUGGGAA |
| 6 | hsa-miR-23a | AUCACAUUGCCAGGGAUUUCC |
| 7 | hsa-miR-125a-5p | UCCCUGAGACCCUUUAACCUGUGA |
| 8 | hsa-miR-512-1 | CACUCAGCCUUGAGGGCACUUUC |
| | (hsa-miR-512-5p) | |
| 9 | hsa-miR-129-1 | CUUUUUGCGGUCUGGGCUUGC |
| | (hsa-miR-129-5p) | |
| 10 | hsa-miR-498 | UUUCAAGCCAGGGGGCGUUUUUC |
| 11 | hsa-miR-22 | AAGCUGCCAGUUGAAGAACUGU |
| 12 | hsa-miR-525-5p | CUCCAGAGGGAUGCACUUUCU |
| 13 | hsa-miR-425 | AAUGACACGAUCACUCCCGUUGA |
| 14 | hsa-miR-638 | AGGGAUCGCGGGCGGGUGGCGGCCU |
| 15 | hsa-miR-602 | GACACGGCCGACAGCUGCGGCCC |
| 16 | hsa-miR-193a-5p | UGGGUCUUUGCGGGCGAGAUGA |
| 17 | hsa-miR-371-5p | ACUCAAACUGUGGGGGCACU |
| 18 | hsa-miR-21* | CAACACCAGUCGAUGGGCUGU |
| 19 | hsa-miR-24-1 | UGGCUCAGUUCAGCAGGAACAG |
| | (hsa-miR-24) | |
| 20 | | CGGCGGCGGCGGCGGCGGCUGU |
| 21 | hsa-miR-16-1 | UAGCAGCACGUAAAUAUUGGCG |
| | (hsa-miR-16) | |
| 22 | hsa-miR-373* | ACUCAAAAUGGGGGCGCUUUCC |
| 23 | hsa-miR-572 | GUCCGCUCGGCGGUGGCCCA |
| 24 | hsa-miR-501-3p | AAUGCACCCGGGCAAGGAUUCU |
| 25 | | AAUGUGUAGCAAAAGACAGAAU |
| 26 | hsa-miR-29a | UAGCACCAUCUGAAAUCGGUUA |
| 27 | hsa-miR-146a | UGAGAACUGAAUUCCAUGGGUU |
| 28 | hsa-miR-671-5p | AGGAAGCCCUGGAGGGGCUGGAG |
| 29 | hsa-miR-623 | AUCCCUUGCAGGGGCUGUUGGGU |
| 30 | hsa-miR-185* | AGGGGCUGGCUUUCCUCUGGUC |
| 31 | hsa-miR-744 | UGCGGGGCUAGGGCUAACAGCA |
| 32 | hsa-miR-193a-3p | AACUGGCCUACAAAGUCCCAGU |
| 33 | | GCGGCGGCGGCGGAGGCUGCUG |
| 34 | hsa-let-7i | UGAGGUAGUAGUUUGUGCUGUU |
| 35 | hsa-miR-492 | AGGACCUGCGGGACAAGAUUCUU |
| 36 | hsa-miR-557 | GUUUGCACGGGUGGGCCUUGUCU |

TABLE 2

| Seq-ID | miRBase | Sequence 5′-3′ |
|---|---|---|
| 37 | hsa-miR-30b | UGUAAACAUCCUACACUCAGCU |
| 38 | hsa-miR-26a-1 | UUCAAGUAAUCCAGGAUAGGCU |
| | (hsa-miR-26a) | |
| 39 | hsa-miR-770-5p | UCCAGUACCACGUGUCAGGGCCA |
| 40 | hsa-miR-30a | UGUAAACAUCCUCGACUGGAAG |
| 41 | hsa-miR-378 | ACUGGACUUGGAGUCAGAAGG |
| 42 | | AAAAGCUGAGUUGAGAGGG |
| 43 | hsa-miR-185 | UGGAGAGAAAGGCAGUUCCUGA |
| 44 | hsa-let-7b | UGAGGUAGUAGGUUGUGUGGUU |
| 45 | hsa-miR-30b* | CUGGGAGGUGGAUGUUUACUUC |
| 26 | hsa-miR-29a | UAGCACCAUCUGAAAUCGGUUA |
| 27 | hsa-miR-146a | UGAGAACUGAAUUCCAUGGGUU |
| 28 | hsa-miR-671-5p | AGGAAGCCCUGGAGGGGCUGGAG |
| 29 | hsa-miR-623 | AUCCCUUGCAGGGGCUGUUGGGU |
| 30 | hsa-miR-185* | AGGGGCUGGCUUUCCUCUGGUC |
| 31 | hsa-miR-744 | UGCGGGGCUAGGGCUAACAGCA |
| 32 | hsa-miR-193a-3p | AACUGGCCUACAAAGUCCCAGU |
| 33 | | GCGGCGGCGGCGGAGGCUGCUG |
| 34 | hsa-let-7i | UGAGGUAGUAGUUUGUGCUGUU |
| 35 | hsa-miR-492 | AGGACCUGCGGGACAAGAUUCUU |
| 36 | hsa-miR-557 | GUUUGCACGGGUGGGCCUUGUCU |

Accordingly, in one aspect the invention relates to the use of one or more micro RNAs comprising a sequence that is essentially homologous to a sequence selected from the group consisting of SEQ IDs 1 to 45 as markers of the functional state of a dendritic cell.

Specifically, in one aspect the invention relates use of one or more micro RNAs comprising a sequence that has at least 70% identity to a nucleic acid sequence selected from the group consisting of SEQ IDs 1 to 45 as markers of the functional state of a dendritic cell, or a dendritic cell population, wherein the differential regulation of the one or more miRNAs is used to indicate the functional state of the dendritic cells.

Specifically, the expression profile of the dendritic cell, or a dendritic cell population, the phenotype of which is to be assayed, is compared to an expression profile of a standard population of dendritic cells. The standard is preferably obtained from a population having a known phenotype. Most preferably the standard is obtained from a population of immature dendritic cells. Preferably, the standard dendritic cell or population of dendritic cells is a dendritic cell or population of dendritic cells originating from the same source (same donor) as the dendritic cell, or the dendritic cell population, the phenotype of which is to be assayed.

More specifically the present inventors found that several micro RNA molecules are differentially expressed in immature versus immunogenic dendritic cells. The micro RNA expression profile of such differentially expressed micro RNA molecules can be used to distinguish between immature and immunogenic dendritic cells.

Accordingly, in one aspect the invention relates to the use of one or more micro RNAs comprising a sequence that is essentially homologous to a sequence selected from the group consisting of SEQ IDs 1 to 36 as markers to distinguish between immature and immunogenic dendritic cells. Particularly preferred sequences were shown to be sequences selected from the group consist of SEQ ID 1, 4, 5, 7, 9, 18, 26, 27, 28, 29, 30 and 31. Even more particularly preferred sequences were shown to be sequences selected from the group consist of SEQ ID 1, 7, 26 and 27.

Even more specifically the present inventors found that several micro RNA molecules are differentially expressed in immature versus tolerogenic dendritic cells. The micro RNA expression profile of such differentially expressed micro RNA molecules can be used to distinguish between immature and tolerogenic dendritic cells.

Accordingly, in one aspect the invention relates to the use of one or mare micro RNAs comprising a sequence that is essentially homologous to a sequence selected from the group consisting of SEQ IDs 26 to 45 as markers to distinguish between immature and tolerogenic dendritic cells. Particularly preferred sequences were shown to be sequences selected from the group consist of SEQ ID 26, 27, 32, 34, 37, 39, 41 and 42. Even more particularly preferred sequences were shown to be sequences selected from the group consist of SEQ ID 27 and 41.

Even more specifically the present inventors found that several micro RNA molecules are differentially expressed in both immature versus immunogenic dendritic cells and immature versus tolerogenic dendritic cells. The micro RNA expression profile of such differentially expressed micro RNA molecules can be used to distinguish between immature and differentiated dendritic cells (both immunogenic and tolerogenic dendritic cells).

Accordingly, in one aspect the invention relates to the use of one or more micro RNAs comprising a sequence that is essentially homologous to a sequence selected from the group consisting of SEQ IDs 26 to 36 as markers to distinguish between immature dendritic cells and tolerogenic or immunogenic dendritic cells

In a preferred aspect of the invention the sequence used comprise a nucleic acid sequence which has at least 70% identity to the nucleic acid sequence of the reference sequence. More specifically, the sequence used comprise a nucleic acid sequence which has at least 75%, such as at least 76, 77, 78, 79, 80, 81, 82, 83, 84, 85, 86, 87, 88 or 89% identity to the reference sequence. Even more preferably, the sequence used comprise a nucleic acid sequence which has at least 90%, such as at least 91, 92, 93, 94, 95, 96, 97, 98, 99, 100% identity to the reference sequence.

In all of the above aspects of the invention it is even evident that the use of two or more micro RNAs will be more useful as the reliability of the result is strengthened and potential errors, for example due to intersample variation, is avoided. Furthermore, the simultaneous use of more than one micro RNA may facilitate the ability to differentiate immature, immunogenic and tolerogenic dendritic cells in a more reliable way, and to do so in as single assay, whereby the general handling becomes easier.

Accordingly, in a preferred aspect the invention relates to the simultaneous use of two or more micro RNAs. In a more preferred aspect the invention relates to the simultaneous use of two or more micro RNAs comprising a sequence that is essentially homologous to a sequence selected from the group consisting of SEQ IDs 1 to 45 as markers of the functional state of a dendritic cell. In an even more preferred aspect the invention relates to the use of three or more micro RNAs comprising a sequence that is essentially homologous to a sequence selected from the group consisting of SEQ IDs 1 to 45 as markers of the functional state of a dendritic cell.

The differentially expressed micro RNAs and/or a micro RNA expression profile or one or more thereof, preferably two or more thereof and even more preferably 3 or more thereof, may thus be used to monitor the state of therapeutic compositions comprising dendritic cells.

Dendritic cells may vary among different mammal species. In a preferred aspect the dendritic cells used in the invention are of human origin.

One object of the invention was to obtain a way of monitoring therapeutic compositions comprising dendritic cells. Accordingly, in one aspect the micro RNAs are used for monitoring the functional state of dendritic cells in vaccine compositions comprising dendritic cells, the so-called “dendritic cell-based vaccine”. Especially suited in this context are also compositions, wherein the dendritic cells are used therapeutically for adoptive transfer.

In relation to this aspect, the invention is further directed at a method for producing a quality-controlled composition comprising dendritic cells, wherein the functional state of the dendritic cells in the composition is monitored, the method comprising the steps of:
a) extracting nucleic acids from the dendritic cells from the composition,
b) monitoring the micro RNA expression profile of one or more micro RNAs comprising a sequence that is essentially homologous to a sequence selected from the group consisting of SEQ IDs 1 to 45 extracted in step a), and
c) comparing the micro RNA expression profile obtained in b) with a standard.

In one aspect, the invention is directed at a method of producing a quality-controlled composition comprising dendritic cells, wherein the functional state of the dendritic cells in the composition is monitored, the method comprising the steps of:
a) extracting nucleic acids from the dendritic cells from the composition,
b) monitoring the micro RNA expression profile of one or more micro RNAs comprising a sequence that is essentially homologous to a sequence selected from the group consisting of SEQ IDs 1 to 36 extracted in step a), and
c) comparing the micro RNA expression profile obtained in b) with a standard.

In one aspect, the invention is directed at a method of producing a quality-controlled composition comprising dendritic cells, wherein the functional state of the dendritic cells in the composition is monitored, the method comprising the steps of:
a) extracting nucleic acids from the dendritic cells from the composition,
b) monitoring the micro RNA expression profile of one or more micro RNAs comprising a sequence that is essentially homologous to a sequence selected from the group consisting of SEQ IDs 26 to 45 extracted in step a), and
c) comparing the micro RNA expression profile obtained in b) with a standard.

In one aspect, the invention is directed at a method of producing a quality-controlled composition comprising dendritic cells, wherein the functional state of the dendritic cells in the composition is monitored, the method comprising the steps of:
a) extracting nucleic acids from the dendritic cells from the composition,
b) monitoring the micro RNA expression profile of one or more micro RNAs comprising a sequence that is essentially homologous to a sequence selected from the group consisting of SEQ IDs 26 to 36 extracted in step a), and
c) comparing the micro RNA expression profile obtained in b) with a standard.

Extraction of nucleic acids may be performed using any of the numerous methods which are well known in the art. The monitoring of the micro RNA expression profile accordingly may be performed by any of the numerous methods which are well known in the art, specifically PCR and preferably real-time PCR, blotting techniques, microarray measurements etc.

In one embodiment the standard is a “universal standard”, where the standard is a total RNA extract with a known concentration of total RNA containing the absolute levels of the one or more micro RNAs according to the invention, verified to be applicable and reliable as specific marker of a specific functional state disregarding any intersample variation. Alternatively, the standard may be a “relative standard”, e.g. an RNA extract from a dendritic cell standard derived from immature dendritic cells of the same origin (same source) as the immunogenic and tolerogenic cells present in the respective composition to be monitored. Preferably the standard is a miRNA profile obtained from a total RNA extract from a population of dendritic cells having an established phenotype (immature, tolerogenic or immunogenic). In a preferred embodiment the standard is obtained form a population of immature dendritic cells. In another preferred embodiment the standard is obtained from a population of dendritic cells originating from the same source, e.g. same donor, as the dendritic cells the phenotype of which is to be established.

The invention further relates to monitored compositions obtainable by a process according to the above.

In another aspect the invention relates to an in vitro system for screening for immunomodulating compounds. The in vitro screening model should serve as a method by which immunomodulating compounds and microorganisms can be assessed for their immunoregulatory properties. This can either be immune stimulation analysed by a compound of interest, e.g. induction of hsa-miR-155 by a TLR-agonist, cytokine, cytokine-derivative or similar, or suppression of a pro-inflammatory cocktail, e.g. the ability of an anti-inflammatory compound like a drug, drug candidate, microorganism or similar, to suppress the induction of a pro-inflammatory miRNA like miRNA-155 or 146, when the anti-inflammatory compounds are added prior to addition of a pro-inflammatory compound (e.g. LPS).

The use according to the invention may thus further be directed towards a method for in vitro screening for immunomodulating compounds. In response to immunomodulatory compounds, the level of expression of a micro RNA according to the above may be altered, whereby immunomodulatory effect of sample compounds may be identified in an in vitro model system.

Such in vitro model systems should preferably comprise dendritic cells at immature, immunogenic or tolerogenic states exposed to test substances potentially affecting the functional state of the dendritic cell.

Thus, in one aspect the invention relates to a method for in vitro screening for compounds having immunomodulatory effect, comprising the steps of:
a) providing a test population of immature dendritic cells,
b) producing from the test population a micro RNA expression profile of at least one micro RNA comprising a sequence that is essentially homologous to a sequence selected from the group consisting of SEQ IDs 1 to 45,
c) contacting the population of dendritic cells with a test compound,
d) producing a micro RNA expression profile as in step b) and,
e) comparing the expression profiles obtained in step b) and step d), where a significant difference in profile will be indicative of immunomodulatory effect of the test compound.

In one embodiment of this method the micro RNAs comprise a sequence that is essentially homologous to a sequence selected from the group consisting of SEQ IDs 1 to 36. In another embodiment of this method the micro RNAs comprise a sequence that is essentially homologous to a sequence selected from the group consisting of SEQ IDs 26 to 45. In another embodiment of this method the micro RNAs comprise a sequence that is essentially homologous to a sequence selected from the group consisting of SEQ IDs 26 to 36.

As above, in this aspect of the invention it is beneficial to use at least two different micro RNAs or even at least three different micro RNAs, preferably wherein the at least two and preferably at least three different micro RNA sequences are selected from the group consisting of SEQ IDs 1 to 45.

### Examples

Generation of Immature, Mature and Tolerogenic Dendritic Cells

Dendritic cells (DC) were prepared using a standard method described elsewhere for generation of DC from peripheral blood monocytes. Typically, the preparation was made from buffy coat obtained from the blood bank. Briefly, peripheral blood mononuclear cells were isolated from buffy coats by standard density gradient methods, Monocytes were then isolated by plastic adhesion and cultured in the presence of GM-CSF (100 ng/mL) and IL-4 (50 ng/mL) for 7 days. The resulting semi-adherent cells were collected for further analysis. These cells are termed “immature DC” or “IDC”.

Immunogenic dendritic cells were generated following the process as described above for immature dendrite cells. However, on day 6 of the culture, a standard DC maturation cocktail comprising TNFα, IL-1β, IL-6 and PGE₂ was added. On day 7 of this culture the resulting cells are termed “mature DC' or “immunogenic DC” or “mDC”.

Tolerogenic dendritic cells were generated according to the process as described above for immunogenic dendritic cells. However, on days 0, 3 and 5 of the culture, 100 ng/mL 1α,25-dihydroxyvitamin D₃ was added to the culture. The resulting cells on day 7 of this culture are termed “tolerogenic DC” or “tolDC”.

The phenotype of day 7 DC was determined by FACS analysis. Cells were labelled using directly conjugated antibodies to HLA-DR/DP/DQ, CD40, CD80, CD86, CCR7 and CD83. Appropriate isotype controls were used. Samples were analysed using FAC-SCalibur Flow Cytometer (Beckton Dickinson) and CELLQuest software (Beckton Dickinson).

The result of a representative experiment is shown in Table 3.

TABLE 3
Expression of DC surface molecules. Values shown
are mean fluorescence intensity. The data shown
below are representative of twelve experiments.

| | HLA-D | CD40 | CD80 | CD86 | CCR7 | CD83 |
|---|---|---|---|---|---|---|
| iDC | 1004.1 | 80.5 | 4.3 | 44.5 | 3.1 | 3.5 |
| mDC | 2508.3 | 145.2 | 20.5 | 861.8 | 43.9 | 102.1 |
| toIDC | 544.8 | 72.6 | 11.2 | 280.3 | 5.9 | 5.0 |

TABLE 4
Cell supernatant was collected from day 7 DC culture and the
levels of IL-12p70 and IL-10 were analysed by sandwich ELISA.
The data shown in Table 4 are representative of twelve experiments.

| | IL-12p70 | IL-10 |
|---|---|---|
| iDC | 8.95 ± 0.06 | 8.65 ± 0.17 |
| mDC | 34.97 ± 0.77 | 33.30 ± 7.12 |
| tolDC | 6.50 ± 0.00 | 49.22 ± 6.31 |

The phenotypes of immature DC and tolerogenic DC resemble in that they have relatively low expression of T cell co-stimulatory molecules such as CD40, CD80 and CD86. Other maturation-associated markers such as CD83 and CCR7 are enhanced in mature DC, but not in immature or tolerogenic DC.

MiRNA profiles of immature, mature and tolerogenic dendritic cells were determined. A miRCURY™ LNA micro RNA array ver. 8.1 (Exiqon NS, Denmark) was used to determine miRNA profiles of populations of dendritic cells (DC) with specific phenotypic characteristics. This microarray comprises a selection of 1476 probes specific for miRNAs in different organisms including 474 probes targeting published (miRBase ver. 8.1) human miRNAs and 150 probes targeting unpublished human miRNA sequences. All probes are represented in quadruplicate on the array.

### Example 1

### Identification of Differentially Expressed Micro RNAs in Mature Immunogenic DC Relative to Immature DC

To identify miRNAs significantly differentially expressed in mature immunogenic DC compared to the immature DC phenotype (reference), dual-labelling hybridisation experiments were conducted using batches of DC from 5 donors (Donor 1, BC#41, BC#43, BC#62 and BC#63).

For each donor the total RNA extracted from immature DC and mature DC was labelled with fluorescent dyes using the miRCURY LNA Array labelling kit (Exiqon). To perform dual labelling hybridisation, RNA from immature DC was labelled with Hy5 (red label) and RNA from mature DC was labelled with Hy3 (green label). To correct for dye biases, array hybridisations were also conducted with RNA labelled vice versa, i.e. immature DC labelled with Hy3 and mature DC and with Hy5.

Two micrograms of total RNA was labelled according to kit-manufacturers instructions and mixed according to the combinations outlined in Table 5 prior to hybridisation to microarray.

TABLE 5
List of combinations of the labelled samples from
the 5 donors used in microarray hybridisations to
the miRCURY LNA micro RNA array

| Array | Total RNA labeled with | Total RNA labeled with |
|---|---|---|
| hybridisation | Hy3 | Hy5 |
| Slide 1 | Donor 1 - immature DC | Donor 1 - mature DC |
| Slide 2 | Donor 1 - mature DC | Donor 1 - immature DC |
| Slide 5 | BC#41 - immature DC | BC#41 - mature DC |
| Slide 6 | BC#41 - mature DC | BC#41 - immature DC |
| Slide 7 | BC#43 - immature DC | BC#43 - mature DC |
| Slide 8 | BC#43 - mature DC | BC#43 - immature DC |
| Slide 9 | BC#62 - immature DC | BC#62 - mature DC |
| Slide 11 | BC#63 - mature DC | BC#63 - immature DC |
| Slide 13 | BC#62 - mature DC | BC#62 - immature DC |
| Slide 15 | BC#63 - immature DC | BC#63 - mature DC |

Microarray hybridisation and subsequent washes were performed according to optimised protocols using a HS 400 Pro Hybridization Station (Tecan). After hybridisation and washes, the dried slides were scanned in an ArrayWoRx white-light CCD-based scanner (Applied Precision, Issaquah, Wash.) using a 0.5 s exposure time in the Cy3 channel (corresponding to detection of Hy3-signal) and a 2 a exposure time in the Cy5 channel (corresponding to detection of Hy5-signal) at a 10 μm resolution. The resulting images were transformed into 16-bit gray scale TIFF-images and imported into ImaGene 8.0 (BioDiscovery Inc.) for further processing.

In ImaGene a microarray grid identifying each spot based on the information GAL-file supplied by the manufacturer of the microarray (Exiqon) was defined, The signal intensity from each channel (Cy5 and Cy3) of each spot was determined as was the local background signal of each spot. Poor spots were flagged and left out in the subsequent analysis. The resulting files with calculated spot intensities and background measurements from all of the donors were compiled and analysed further using GeneSight-Lite 4.1.6 (BioDiscovery Inc.).

In GeneSight-Lite a ratio analysis was conducted based on two different normalisation methods. One analysis employed Lowess normalisation and another employed divison by mean signal intensity normalisation. The calculated ratio values for all micro RNAs were expressed as the ratio between the normalised signal intensity in the mature DC and the normalised intensity of the reference immature DC.

Ratio values from all experiments (slides 1, 2, 5, 6, 7, 8, 9, 11, 13 and 15) were compiled using both Lowess and divison by mean signal normalisation, and statistical analyses were performed to identify miRNAs with significantly differential expression in the mature DC compared to immature DC. Prior to analysis ratio values were log₂ transformed and a one-class t-test assuming unequal variance between the samples was conducted to identify miRNAs that had ratio values significantly (P<0.05) different from 0 (equal to the untransformed ratio value 1). Thus, a value above 0 indicates higher expression in the mature DC than in the immature DC and a value below 0 indicates lower expression in the mature DC than in the immature DC. In addition a more rigid statistical analysis was performed using the Significance Analysis of Microarray (SAM), which includes a correction of multiple testing commonly used in microarray analysis.

In Table 6 the list of micro RNAs that were identified as significantly differentially expressed with both normalisation methods is depicted.

TABLE 6

| | | | | | | | |
|---|---|---|---|---|---|---|---|
| | | | | | | SAM | SAM |
| Seq-ID | miRBase name | Sequence 5′-3′ | M/IM | P (L) | P (DM) | (L) | (DM) |

| | | | | | | | |
|---|---|---|---|---|---|---|---|
| 1 | hsa-miR-27a | UUCACAGUGGCUAAGUUCCGC | 0.69 | 2.70E−05 | 5.46E−05 | + | + |
| 2 | hsa-miR-231a | AUCACAUUGCCAGGGAUUACC | 0.51 | 1.87E−04 | 1.07E−04 | + | |
| 3 | hsa-miR-27b | UUCACAGUGGCUAAGUUCUGC | 0.58 | 2.70E−04 | 0.0111 | + | |
| 4 | hsa-miR-155 | UUAAUGCUAAUCGUGAUAGGGGU | 2.64 | 2.71E−04 | 4.71E−06 | + | + |
| 5 | hsa-miR-202 | AGAGGUAUAGGGCAUGGGAA | −0.53 | 5.21E−04 | 0.003 | + | + |
| 6 | hsa-miR-23a | AUCACAUUGCCAGGGAUUUCC | 0.44 | 9.94E−04 | 3.88E−04 | | |
| 7 | hsa-miR-125a-5p | UCCCUGAGACCCUUUAACCUGUGA | 0.82 | 0.0013 | 5.98E−04 | + | + |
| 8 | hsa-miR-512-1 | CACUCAGCCUUGAGGGCACUUUC | −0.4 | 0.0027 | 0.0059 | | |
| | (hsa-miR-512-5p) | | | | | | |
| 9 | hsa-miR-129-1 | CUUUUUGCGGUCUGGGCUUGC | −0.79 | 0.0035 | 0.0015 | + | + |
| | (hsa-miR-129-5p) | | | | | | |
| 10 | hsa-miR-498 | UUUCAAGCCAGGGGGCGUUUUUC | −0.45 | 0.0049 | 0.0023 | | + |
| 11 | hsa-miR-22 | AAGCUGCCAGUUGAAGAACUGU | 0.5 | 0.0095 | 0.0076 | | |
| 12 | hsa-miR-525-5p | CUCCAGAGGGAUGCACUUUCU | −0.29 | 0.0017 | 0.01 | | |
| 13 | hsa-miR-425 | AAUGACACGAUCACUCCCGUUGA | 0.48 | 0.0026 | 0.0111 | | |
| 14 | hsa-miR-638 | AGGGAUCGCGGGCGGGUGGGGGCCU | −0.55 | 0.0048 | 0.0165 | | + |
| 15 | hsa-miR-602 | GACACGGGCGACAGCUGCGGCCC | −0.48 | 0.0126 | 0.0038 | | |
| 16 | hsa-miR-193a-5p | UGGGUCUUUGCGGGCGAGAUGA | −0.5 | 0.0172 | 0.0136 | | + |
| 17 | hsa-miR-261-5p | ACUCAAACUGUGGGGGCACU | −0.46 | 0.018 | 0.0203 | | |
| 18 | hsa-miR-21* | CAACACCAGUCGAUGGGCUGU | −0.77 | 0.0183 | 0.0128 | + | + |
| 19 | hsa-miR-24-1 | UGGCUCAGUUCAGGAGGAACAG | 0.38 | 0.0244 | 0.0285 | | |
| | (hsa-miR-24) | | | | | | |
| 20 | | CGGCGGCGGCGGCGGCGGCUGU | −0.63 | 0.0264 | 0.0119 | | |
| 21 | hsa-miR-16-1 | UAGCAGCACGUAAAUAUUGGCG | 0.26 | 0.0293 | 0.0427 | | |
| | (hsa-miR-16) | | | | | | |
| 22 | hsa-miR-373* | ACUCAAAAUGGGGGCGCUUUCC | −0.51 | 0.0299 | 0.0144 | | + |
| 23 | hsa-miR-572 | GUCCGCUCGGCGGUGGCCCA | −0.45 | 0.0368 | 0.0109 | | |
| 24 | hsa-miR-501-3p | AAUGCACCCGGGCAAGGAUUCU | −0.22 | 0.0396 | 0.0165 | | |
| 25 | | AAUGUGUAGCAAAAGACAGAAU | −0.54 | 0.0432 | 0.0225 | | + |
| 26 | hsa-miR-29a | UAGCACCAUCUGAAAUCGGUUA | 0.94 | 1.62E−04 | 7.01E−04 | + | + |
| 27 | hsa-miR-146a | UGAGAACUGAAUUCCAUGGGUU | 1.55 | 3.02E−04 | 3.46E−04 | + | + |
| 28 | hsa-miR-671-5p | AGGAAGCCCUGGAGGGGCUGGAG | −0.86 | 0.0016 | 0.0045 | + | + |
| 29 | hsa-miR-623 | AUCCCUUGCAGGGGCUGUUGGGU | −0.73 | 0.0023 | 0.0049 | + | + |
| 30 | hsa-miR-185* | AGGGGCUGGCUUUCCUCUGGUC | −0.79 | 0.0046 | 7.57E−04 | + | + |
| 31 | hsa-miR-744 | UGCGGGGCUAGGGCUAACAGCA | −0.92 | 0.005 | 0.0027 | + | + |
| 32 | hsa-miR-193a-3p | AACUGGCCUACAAAGUCCCAGU | 0.69 | 0.0107 | 0.0158 | + | |
| 33 | | GCGGCGGCGGCGGAGGCUGCUG | −0.55 | 0.0033 | 0.0164 | | + |
| 34 | hsa-let-7i | UGAGGUAGUAGUUUGUGCUGUU | 0.42 | 0.0242 | 0.0382 | | |
| 35 | hsa-miR-492 | AGGACCUGCGGGACAAGAUUCUU | −0.71 | 0.0107 | 0.0143 | | + |
| 36 | hsa-miR-557 | GUUUGCACGGGUGGGCCUUGUCU | −0.49 | 0.0423 | 0.0166 | | + |

Table 6 shows a list of micro RNAs significantly differentially expressed in mature immunogenic DC relative to immature immunogenic DC. In all cases, except for SEQ-ID 20, 25 and 33, the official names according to the miRBase version 12.0 are shown. In parentheses, in some cases, the name referred to in the miRBase version 10.0 is shown. SEQ-IDs 20, 25 and 33 represent sequences of micro RNAs proprietary to Exiqon A/S that have not yet been made public in the miRBase. The M/IM values represent the average log₂ transformed ratios of the two ratio values calculated based on the two different normalisation methods (L: Lowess, DM: Divide by mean density). P (L) and P (DM) respectively indicate the resulting P-values of the performed t-test associated with either L or DM normalisation. The last two columns shows if the determined differential expression could pass a more rigid statistical analysis using SAM to correct for multiple testing using either the Lowess normalised (L) or the divide-by-mean normalized (DM) data. A ‘+’ indicates that the differential expression of the given microRNA passed the more rigid statistical analysis.

Thus a “+” show that the specified sequence is differentially expressed in immunogenic DC compared to immature DC, and that the differential expression is statistically significant by that statistic analysis. A positive T/IM value indicates that the expression of the specified sequence is expressed at a higher level in immunogenic DC compared to immature DC, whereas a negative T/IM value indicates that the expression of the specified sequence is expressed at a lower level in immunogenic DC compared to immature DC.

A total of 36 different micro RNAs differentially expressed in mature immunogenic DC relative to immature DC were identified. Of those 14 were upregulated in the range of 1.3-6.2 fold and 22 were downregulated in the range of 1.2-1.9 fold in the immunogenic DC compared to immature DC.

More specifically, the sequences that were upregulated in immunogenic DC compared to immature DC were seq ID nr. 1, 2, 3, 4, 6, 7, 11, 13, 19, 21, 26, 27, 32 and 34. The sequences that were downregulated in immunogenic DC compared to immature DC were seq ID nr. 5, 8, 9, 10, 12, 14, 15, 16, 17, 18, 20, 22, 23, 24, 25, 28, 29, 30, 31, 33, 35 and 36.

### Example 2

### Identification of Differentially Expressed Micro RNAs in Tolerogenic DC Relative to Immature DC

To identify miRNAs significantly differentially expressed in tolerogenic DC relative to the immature DC phenotype, dual labelling hybridisation experiments were conducted using batches of DC from 3 donors (Donor 1, BC#62 and BC#63).

For each donor total RNA extracted from immature DC and tolerogenic DC were labelled with fluorescent dyes using the miRCURY LNA Array labelling kit. To perform dual labelling hybridisation, RNA from immature DC was labelled with Hy5 (red label) and RNA from tolerogenic DC was labelled with Hy3 (green label). To correct for dye biases, array hybridisations were also conducted with RNA labelled vice versa, i.e. immature DC labeled with Hy3 and tolerogenic DC with Hy5.

Two micrograms of total RNA was labelled according to kit manufacturers instructions and mixed according to the combinations outlined in Table 7 prior to hybridisation to microarray.

TABLE 7
List of combinations of the labeled samples from
the 3 donors used in microarray hybridisations
to the miRCURY LNA micro RNA array.

| Array | Total RNA labeled | Total RNA labeled |
|---|---|---|
| hybridisation | with Hy3 | with Hy5 |
| Slide 3 | Donor 1 - immature DC | Donor 1 - tolerogenic DC |
| Slide 4 | Donor 1 - tolerogenic DC | Donor 1 - immature DC |
| Slide 10 | BC#62 - tolerogenic DC | BC#62 - immature DC |
| Slide 12 | BC#63 - immature DC | BC#63 - tolerogenic DC |
| Slide 14 | BC#62 - immature DC | BC#62 - tolerogenic DC |
| Slide 16 | BC#63 - tolerogenic DC | BC#63 - immature DC |

Microarray hybridisation and subsequent washes were performed according to optimised protocols using a HS 400 Pro Hybridization Station (Tecan). After hybridisation and washes the dried slides were scanned in an ArrayWoRx white-light CCD-based scanner (Applied Precision, Issaquah, Wash.) using a 0.5 s exposure time in the Cy3 channel (corresponding to detection of Hy3-signal) and a 2 s exposure time in the Cy5 channel (corresponding to detection of Hy5-signal) at a 10 μm resolution. The resulting images were transformed into 16-bit gray scale TIFF-images and imported into ImaGene 8.0 (BioDiscovery Inc.) for further processing. In ImaGene a microarray grid identifying each spot based on the information GAL-file supplied by the manufacturer of the microarray (Exiqon) was defined. The signal intensity from each channel (Cy5 and Cy3) of each spot was determined as was the local background signal of each spot. Poor spots were flagged and left out in the subsequent analysis. The resulting files with calculated spot intensities and background measurements from all of the donors were compiled and analysed further using GeneSight-Lite 4.1.6 (BioDiscovery Inc.). In GeneSight-Lite a ratio analysis was conducted based on two different normalisation methods. One analysis employed Lowess normalisation and another employed divison by mean signal intensity normalisation. The calculated ratio values for all micro RNAs were expressed as the ratio between the normalised signal intensity in the tolerogenic DC and the normalised intensity of the reference immature DC.

Ratio values from all experiments (slides 3, 4, 10, 12, 14 and 16) were compiled using both Lowess and divison by mean signal normalisation, and statistical analyses were performed to identify miRNAs with significantly differential expression in the tolerogenic DC compared to immature DC. Prior to analysis, ratio values were log₂ transformed and a one-class t-test assuming unequal variance between the samples was conducted to identify miRNAs that had ratio values significantly (P<0.05) different from 0 (equal to the untransformed ratio value 1). Thus, a value above 0 indicates higher expression in the tolerogenic DC than in the immature DC, and a value below 0 indicates lower expression in the tolerogenic DC than in the immature DC. In addition a more rigid statistical analysis was performed using the Significance Analysis of Microarray (SAM), which includes a correction of multiple testing commonly used in microarray analysis.

In Table 8 the list of micro RNAs that were identified as significantly differentially expressed with both normalisation methods is depicted.

TABLE 8

| | | | | | | SAM | SAM |
|---|---|---|---|---|---|---|---|
| Seq-ID | miRBase | Sequence 5′-3′ | T/IM | P (L) | P (DM) | (L) | (DM) |
| 37 | hsa-miR-30b | UGUAAACAUCCUACACUCAGCU | 0.51 | 0.0013 | 2.76E−04 | + | + |
| 38 | hsa-miR-26a-1 | UUCAAGUAAUCCAGGAUAGGCU | 0.28 | 0.0158 | 0.0273 | + | |
| | (hsa-miR-26a) | | | | | | |
| 39 | hsa-miR-770-5p | UCCAGUACCACGUGUCAGGGCCA | 1.73 | 0.0091 | 0.0041 | + | + |
| 40 | hsa-miR-30a | UGUAAACAUCCUCGACUGGAAG | 0.49 | 0.0315 | 0.0107 | | + |
| 41 | hsa-miR-378 | ACUGGACUUGGAGUCAGAAGG | 0.64 | 0.0246 | 0.0213 | + | + |
| 42 | | AAAAGCUGAGUUGAGAGGG | 0.6 | 0.0117 | 0.0267 | + | + |
| 43 | hsa-miR-185 | UGGAGAGAAAGGCAGUUCCUGA | −0.27 | 0.0191 | 0.0035 | | |
| 44 | hsa-let-7b | UGAGGUAGUAGGUUGUGUGGUU | −0.21 | 0.0254 | 0.0113 | | |
| 45 | hsa-miR-30b* | CUGGGAGGUGGAUGUUUACUUC | −0.19 | 0.0339 | 0.0078 | | |
| 26 | hsa-miR-29a | UAGCACCAUCUGAAAUCGGUUA | 0.51 | 0.0012 | 7.67E−04 | + | + |
| 27 | hsa-miR-146a | UGAGAACUGAAUUCCAUGGGUU | 1.3 | 0.0022 | 0.0075 | + | + |
| 28 | hsa-miR-671-5p | AGGAAGCCCUGGAGGGGCUGGAG | −0.26 | 0.039 | 0.039 | | |
| 29 | hsa-miR-623 | AUCCCUUGCAGGGGCUGUUGGGU | −0.24 | 0.0241 | 0.0068 | | |
| 30 | hsa-miR-185* | AGGGGCUGGCUUUCCUCUGGUC | −0.41 | 0.0455 | 0.0408 | | |
| 31 | hsa-miR-744 | UGCGGGGCUAGGGCUAACAGCA | −0.49 | 0.0129 | 0.0043 | | |
| 32 | hsa-miR-193a-3p | AACUGGCCUACAAAGUCCCAGU | 1.48 | 0.0049 | 2.46E−05 | + | + |
| 33 | | GCGGCGGCGGCGGAGGCUGCUG | −0.19 | 0.0092 | 0.0166 | | |
| 34 | hsa-let-7i | UGAGGUAGUAGUUUGUGCUGUU | 0.46 | 0.0202 | 0.0222 | + | + |
| 35 | hsa-miR-492 | AGGACCUGCGGGACAAGAUUCUU | −0.36 | 0.0239 | 0.0068 | | |
| 36 | hsa-miR-557 | GUUUGCACGGGUGGGCCUUGUCU | −0.41 | 0.0459 | 0.027 | | |

Table 8 shows a list of micro RNAs significantly differentially expressed in tolerogenic DC relative to immature DC. In all cases, except for SEQ-ID 42 and 33, the official names according to the miRBase version 12.0 are shown. In parentheses, in some cases, the name referred to in the miRBase version 10.0 is shown. SEQ-IDs 42 and 33 represent sequences of micro RNAs proprietary to Exiqon A/S that have not yet been made public in the miRBase. The T/IM values represent the average log₂ transformed ratios of the two ratio values calculated based on the two different normalisation methods (L: Lowess, DM: Divide by mean density). P (L) and P (DM) respectively indicate the resulting P-values of the performed t-test associated with either L or DM normalisation. The last two columns shows if the determined differential expression could pass a more rigid statistical analysis using SAM to correct for multiple testing using either the Lowess normalised (L) or the divide-by-mean normalized (DM) data. A ‘+’ indicates that the differential expression of the given microRNA passed the more rigid statistical analysis.

Thus a “+” show that the specified sequence is differentially expressed in tolerogenic DC compared to immature DC, and that the differential expression is statistically significant by that statistic analysis. A positive T/IM value indicates that the expression of the specified sequence is expressed at a higher level in tolerogenic DC compared to immature DC, whereas a negative T/IM value indicates that the expression of the specified sequence is expressed at a lower level in tolerogenic DC compared to immature DC.

A total of 20 different micro RNAs differentially expressed in tolerogenic DC relative to immature DC were identified. Of those, 10 were upregulated in the range of 1.2-3.3 fold and 10 were downregulated in the range of 1.1-1.4 fold in the tolerogenic DC compared to immature DC.

More specifically, the sequences that were upregulated in tolerogenic DC compared to immature DC were seq ID nr. 26, 27, 32, 34, 37, 38, 39, 40, 41 and 42. The sequences that were downregulated in tolerogenic DC compared to immature DC were seq ID nr. 28, 29, 30, 31, 33, 35, 36, 43, 44 and 45.

A common set of micro RNAs are differentially regulated in both tolerogenic and mature DC relative to immature DC. This set of micro RNA (Table 9) comprises a total of 11 micro RNAs that are equally up or downregulated in either mature or tolerogenic DC compared to the immature state of DC.

TABLE 9

| | | | | |
|---|---|---|---|---|
| Seq- | miRBase | | | |
| ID | name | Sequence 5′-3′ | M/IM | T/IM |

| | | | | |
|---|---|---|---|---|
| 26 | hsa-miR-29a | UAGCACCAUCUGAAAUCGGUUA | 0.94 | 0.51 |
| 27 | hsa-miR-146a | UGAGAACUGAAUUCCAUGGGUU | 1.55 | 1.3 |
| 28 | hsa-miR-671-5p | AGGAAGCCCUGGAGGGGCUGGAG | −0.86 | −0.26 |
| 29 | hsa-miR-623 | AUCCCUUGCAGGGGCUGUUGGGU | −0.73 | −0.24 |
| 30 | hsa-miR-185* | AGGGGCUGGCUUUCCUCUGGUC | −0.79 | −0.41 |
| 31 | hsa-miR-744 | UGCGGGGCUAGGGCUAACAGCA | −0.92 | −0.49 |
| 32 | hsa-miR-193a-3p | AACUGGCCUACAAAGUCCCAGU | 0.69 | 1.48 |
| 33 | | GCGGCGGCGGCGGAGGCUGCUG | −0.55 | −0.19 |
| 34 | hsa-let-7i | UGAGGUAGUAGUUUGUGCUGUU | 0.42 | 0.46 |
| 35 | hsa-miR-492 | AGGACCUGCGGGACAAGAUUCUU | −0.71 | −0.36 |
| 36 | hsa-miR-557 | GUUUGCACGGGUGGGCCUUGUCU | −0.49 | −0.41 |

Table 9 shows the common set of 11 micro RNAs that are equally up or downregulated in both mature and tolerogenic DC relative to immature DC. The level of regulation (as log₂ transformed fold change) is indicated in the columns M/IM and T/IM for mature and tolerogenic DC respectively.

More specifically, the sequences that were upregulated in both immunogenic DC and tolerogenic DC compared to immature DC were seq ID nr. 26, 27, 32 and 34. The sequences that were downregulated in both immunogenic DC and tolerogenic DC compared to immature DC were seq ID nr. 28, 29, 30, 31, 33, 35 and 36.

### Example 3

### Validation of hsa-miR-155 Induction in Immunogenic DC Using Real-Time PCR

In Example 1, hsa-miR-155 was shown to be significantly upregulated in immunogenic DC compared to immature DC in 5 different donors. Based on microRNA profiling and ratio analysis the upregulation was determined to be in the average range of 6-fold (see Table 6). To validate this upregulation real-time PCR targeting the hsa-miR-155 was conducted.

The expression of mature miRNAs was assayed using the TaqMan MicroRNA Assays (Applied Biosystems) specific for hsa-miR-155 (P/N: 4373124). The relative expression level of hsa-miR-155 was measured in 5 different immunogenic DC representing the 5 donors (Donor1, BC#41, BC#43, BC#62, and BC#63) by comparison to the expression level in the respective immature DC.

In addition, the hsa-miR-155 expression level was also measured in two batches of tolerogenic DC representing two of the donors (Donor1 and BC#A62).

Each sample was analysed in triplicate.

Briefly, single-stranded c-DNA was synthesised from 10 ng of total RNA by using the looped primers of the TaqMan MicroRNA Assay and the TaqMan MicroRNA Reverse Transcription Kit (Applied Biosystems).

The reactions were incubated first at 16° C. for 30 minutes and then at 42° C. for 30 minutes, ending with an inactivation incubation at 85° C. for 5 minutes.

Each cDNA generated was amplified by real-time PCR using the sequence-specific primers from the TaqMan MicroRNA Assay. Real-time PCR was done using the standard TaqMan MicroRNA Assays protocol on the ABI StepOne Plus real-time PCR machine (Applied Biosystems). The 10 μL PCR included 0.67 μL reverse transcription product, 1× TaqMan Universal PCR Master Mix, No AmpErase UNG (Applied Biosystems), 0.2 μM TaqMan probe, 1.5 μM forward primer and 0.7 μM reverse primer.

The reactions were incubated in a 96-well plate at 95° C. for 10 minutes followed by 40 cycles of 95° C. for 15 seconds and 60° C. for 1 minute.

The level of miRNA expression was measured using Ct (threshold cycle). The Ct is the fractional cycle number at which the fluorescence of each sample passes the fixed threshold. The ΔΔCt method (Livak and Schmittgen, (2001), Methods 25:402-409) for relative quantification of gene expression was used to determine miRNA expression levels relative to an endogenous control and the reference immature DC. To normalise the relative abundance of hsa-miR-155, U6 RNA was used as endogenous control. U6 RNA was detected using the Taq Man MicroRNA Assay for U6 RNA (RNU6B, P/N: 4373381; Applied Biosystems). The ΔCt was calculated by subtracting the Ct of U6 RNA from the Ct of the hsa-miR-155. The ΔΔCt was calculated by subtracting the ΔCt of the reference sample (immature DC) from the ΔCt of each sample. Fold change was generated using the equation 2^{-ΔΔCt}. Table 10 lists the calculated fold changes (corresponding to the 2^{-ΔΔCt} value) of the immunogenic and tolerogenic DC with respect to hsa-miR-155 expression relative to the respective immature DC.

TABLE 10

| Donor | Phenotype | ΔΔCt | 2^{-ΔΔCt} |
|---|---|---|---|
| Donor 1 | immunogenic | −4.16 ± 0.49 | 17.8 (12.7-25.1) |
| Donor 1 | tolerogenic | −0.92 ± 0.02 | 1.89 (1.87-1.92) |
| BC#41 | immunogenic | −1.95 ± 0.24 | 3.9 (3.3-4.6) |
| BC#43 | immunogenic | −4.22 ± 0.14 | 18.6 (16.9-20.5) |
| BC#62 | immunogenic | −2.19 ± 0.07 | 4.6 (4.3-4.8) |
| BC#62 | tolerogenic | 0.41 ± 0.8 | 0.75 (0.71-0.80) |
| BC#63 | immunogenic | −3.58 ± 0.3 | 12.0 (9.7-14.7) |

Table 10 shows a list of calculated fold-changes corresponding to the 2^{-ΔΔCt} value of hsa-miR-155 in either immunogenic or tolerogenic DC phenotypes relative to immature DC of five different donors. In the last column, in parentheses, the fold-change range is indicated as calculated from the standard deviation of the ΔΔCt value shown in column 3.

From the results shown in Table 10 it is clear that there is a general induction of hsa-miR-155 in immunogenic DC with an average 11 fold-change compared to the expression level in immature DC. In the two included tolerogenic phenotypes the average fold-change is only 1.3. Thus, the real-time PCR analysis on hsa-miR-155 has clearly validated the preliminary results obtained using microarray profiling.

### Example 4

### Validation of hsa-miR-155, hsa-miR-146a, hsa-miR-125a-5p and hsa-miR-29a Induction in Immunogenic DC

Additional donors were enrolled in the validation of a selected number of microRNAs using real time PCR analogously to the procedure described in Example 3. By using a total of 12 donors (including donors: BC#41, BC#43, BC#62 and BC#63 from the previous Example 3+ donors: BC#103, BC#106, BC#126, BC#128, BC#130, BC#136, BC#160 and BC#174), characterized phenotypically by surface markers and cytokine profiles (for more details se Example 5), the real-time PCR results as seen in Table 11 was obtained.

TABLE 11

| microRNA SEQ ID | ΔΔCt | P-value | 2^{-ΔΔCt} |
|---|---|---|---|
| 4 (hsa-miR-155) | −1.90 ± 1.32 | 4.25E−04 | 3.73 (1.49-9.32) |
| 27 (hsa-miR-146a) | −1.36 ± 0.49 | 1.03E−06 | 2.57 (1.82-3.61) |
| 7 (hsa-miR-125a-5p) | −0.86 ± 0.67 | 0.0016 | 1.82 (1.14-2.89) |
| 26 (hsa-miR-29a) | −0.35 ± 0.63 | 0.0817 | 1.27 (0.82-1.97) |

Table 11 shows a list of average ΔΔCt values and the corresponding fold-changes calculated with 2^{-ΔΔCt} of hsa-miR-155, hsa-miR-146a, hsa-miR-125a-5p and hsa-mIR-29a in immunogenic DC phenotypes relative to the respective immature DC in 12 different donors. In the third column, the P-value is indicated. The P-value was determined using a one-class t-test to test if the ΔΔCt value of the different microRNAs were significantly different from 0.

From the results shown in Table 11 it is clear that there is a statistically significant (P<0.05) induction of hsa-miR-155, hsa-miR-146a and hsa-miR-125a-5p in immunogenic DC compared to the expression level in immature DC. Although an induction of the fourth microRNA tested (SEQ ID 26=hsa-miR-29a) was observed, this could not be shown to be statistically significantly validated for induction in immunogenic DCs in the current experiment using the number of donors available for this test.

### Example 5

### Determination of miRNA-Markers with the Ability to Distinguish Between Successfully Cocktail Matured DC Batches and Poorly Matured DCs

A group of 12 donors (the same donors as mentioned in Example 4) were matured with the cocktail comprising TNFα, IL-β, IL-6 and PGE₂. Traditional immunogenic maturation markers (HLA-D, CD80, CD83, CD86, CCR7, IL-12p70 and IL-23) were determined and scored for their induction level (Table 12). The total score for each donor's cocktail treated DCs were summed up to reach total scores between 10 and 22 representing poor, i.e. partially or less-differentiated mDCs, and good responders, i.e. fully-differentiated mDCs, respectively.

TABLE 12

| Donor ID | HLA-D | CD83 | CCR7 | CD86 | CD80 | IL12p70 | IL23 | total score |
|---|---|---|---|---|---|---|---|---|
| BC#41 | 1 | 3 | 3 | 3.0 | 2.0 | 2 | 3 | 17 |
| BC#43 | 1.0 | 3.0 | 3.0 | 3.0 | 2.0 | 1 | 3 | 16 |
| BC#62 | 1.0 | 3.0 | 2.0 | 3.0 | 2.0 | 3 | 3 | 17 |
| BC#63 | 1.0 | 3.0 | 2.0 | 3.0 | 2.0 | 4 | 3 | 18 |
| BC#106 | 2.0 | 2.0 | 1.0 | 3.0 | 3.0 | 2 | 3 | 16 |
| BC#126 | 3.0 | 2.0 | 0.0 | 2.0 | 3.0 | 1 | 3 | 14 |
| BC#128 | 3.0 | 3.0 | 3.0 | 3.0 | 4.0 | 3 | 3 | 22 |
| BC#103 | 2.0 | 1.0 | 0.0 | 1.0 | 3.0 | 0 | 3 | 10 |
| BC#130 | 2.0 | 2.0 | 1.0 | 2.0 | 3.0 | 1 | 3 | 14 |
| BC#136 | 3.0 | 2.0 | 0.0 | 3.0 | 3.0 | 2 | 0 | 13 |
| BC#160 | 2.0 | 2.0 | 2.0 | 3.0 | 3.0 | 3 | 3 | 18 |
| BC#174 | 1.0 | 2.0 | 0.0 | 3.0 | 3.0 | 2 | 2 | 13 |

Table 12. Scoring table based on exact levels of the markers indicated in the upper line.

The markers were selected based on the current knowledge of DC maturation, including hall-mark immunogenic secreted cytokines IL-12p70 and IL-23, and membrane expressed co-receptors important for T-cell interaction HLA-D, CD83, CD86 and CD80, and finally the expression level of CCR7 which is important for DC migration to lymph nodes where activation of T-cells occurs. Each donor was scored for the ability to increase the specific marker determined either by ELISA of secreted cytokines into the media or by FACS analyses of the DCs after 24 hr treatment with the maturation cocktail. An overall scoring was made, where 0 was given to DC batches where the level of marker was below the level seen generally for non-cocktail treated cells, 1 was given if the marker was just slightly higher than the overall level of non-cocktail treated cells, 2 was given if the marker was noticeably higher than the overall level of non-cocktail treated cells, 3 was given if the marker was higher than the overall level of non-cocktail treated cells and 4 was given if the marker was much higher than the overall level of non-cocktail treated cells. The total scores for each donor was summed up and shown in the very right column, where the donor with the highest score is considered as having the most suitable maturation status.

Because human monocyte derived DCs can behave in a heterogeneous manner when exposed to maturation cocktails, we wanted to be able to distinguish good responders from poor responding DC-batches, to investigate if the significance of induction of selected microRNAs were different in these two groups. Based on the scoring system presented in Table 12 two donor groups were defined; the first with scores at 14 or below (poor responders) and the second group with a score of 16 or above (good responder group) (Table 13). The level of maturation markers for each donor was expressed as fold increase compared to immature DCs. The miRNA induction levels for each donor group were determined using real-time RT-PCR as described in Examples 3 and 4 and expressed as −ΔΔCt values corresponding to the log₂ transformed fold increase relative to immature DCs (Table 13, right part).

TABLE 13

| Donor ID | HLA-D | CD83 | CCR7 | CD86 | CD80 | IL12p70 | II23 | score |
|---|---|---|---|---|---|---|---|---|
| Good responders | | | | | | | | |
| BC#41 | 1.028761 | 14.58108 | 12.23404 | 5.266289 | 2.679487 | 7.917 | 26.93 | 17 |
| BC#43 | 3.137947 | 11.95726 | 11.225 | 4.868117 | 3.27907 | 1.6 | 81.06667 | 16 |
| BC#62 | 1.7977 | 17.3051 | 9.387097 | 8.844086 | 5.538462 | 4.210526 | 16.9666 | 17 |
| BC#63 | 1.79433 | 61.21429 | 14.63333 | 8.901158 | 4.164859 | 57.5 | 51.57616 | 18 |
| BC#106 | 1.427814 | 11.01887 | 2.176471 | 8.397568 | 6.128571 | 1.142857 | 26.93 | 16 |
| BC#128 | 1.533593 | 42.61702 | 9.440673 | 5.634551 | 8.722846 | 3.5 | 36.37079 | 22 |
| BC#160 | 1.19256 | 1.620455 | 5.240741 | 4.859752 | 3.654088 | 9.89 | 25.73713 | 18 |
| average | 1.701815 | 22.90201 | 9.191052 | 6.681646 | 4.881055 | 12.26577 | 37.93962 | 17.71429 |
| stdev | 0.694736 | 21.09888 | 4.241172 | 1.325902 | 2.088839 | 20.19964 | 21.90719 | 2.058663 |

t-test (different from 0), P value

| | | | | | | | | |
|---|---|---|---|---|---|---|---|---|
| Poor respomders | | | | | | | | |
| BC#103 | 3.248821 | 2.740741 | 1.571429 | 6.192529 | 3.122699 | 0.666667 | 9.464789 | 10 |
| BC#174 | 1.290506 | 10.75439 | 2.935484 | 17.59654 | 3.439189 | 3.75 | 18.84184 | 13 |
| BC#136 | 1.186713 | 10.95238 | 1.546875 | 2.462973 | 4.170543 | 1.727273 | 2.223108 | 13 |
| BC#126 | 2.105618 | 6.1 | 1.4 | 5.692 | 4.201342 | 1.5 | 14.7561 | 14 |
| BC#130 | 1.906988 | 15.71429 | 2.925926 | 4.807851 | 4.095745 | 1.5 | 12.29412 | 14 |
| average | 1.947729 | 9.252359 | 2.075943 | 7.350379 | 3.805904 | 1.828788 | 11.51599 | 12.8 |
| stdev | 0.826214 | 4.980994 | 0.783043 | 5.903937 | 0.493606 | 1.147616 | 6.231711 | 1.643168 |
| t-test, P value | 0.60273 | 0.143793 | 0.003968 | 0.817584 | 0.231574 | 0.221111 | 0.018332 | 0.001048 |

t-test (different from 0), P value

| | miRNA-data | | | |
|---|---|---|---|---|
| Donor ID | miR-155 | miR-146a | miR-125a-5 | miR-29a |
| Good responders | | | | |
| BC#41 | 1.95 | 0.78 | 0.92 | 0.23 |
| BC#43 | 3.87 | 1.15 | 0.75 | 1.07 |
| BC#62 | 1.98 | 1.33 | 1.14 | 0.51 |
| BC#63 | 3.41 | 2.13 | 1.23 | 0.5 |
| BC#106 | 0.94 | 1.61 | 0.47 | −0.09 |
| BC#128 | 3.6 | 2.21 | 1.28 | 1.29 |
| BC#160 | 3.24 | 1.65 | 1.89 | 0.55 |
| average | 2.7129571 | 1.5514286 | 0.962857 | 0.58 |
| stdev | 1.0917832 | 0.5142771 | 0.726079 | 0.470425 |
| t-test (different from 0), P value | 5.94E−04 | 2.06E−04 | 0.012694 | 0.017205 |
| Poor respomders | | | | |
| BC#103 | 0.74 | 1.5 | 0.21 | −0.63 |
| BC#174 | 1.38 | 1.25 | 1.45 | 0.74 |
| BC#136 | 0.85 | 0.77 | | 0.8 |
| BC#126 | 0.06 | 1.13 | 0.2 | −0.76 |
| BC#130 | 0.73 | 0.79 | 0.91 | −0.02 |
| average | 0.752 | 1.088 | 0.6925 | 0.026 |
| stdev | 0.4699681 | 0.3113198 | 0.604559 | 0.734697 |
| t-test, P value | 0.0024013 | 0.0817656 | 0.527785 | 0.186134 |
| t-test [different from 0), P value | 0.02321 | 0.00144 | 0.105866 | 0.94072 |

Based on total scores (Table 12), the donors were divided into two groups with a good cocktail responder group (highest score) and a group responding poorly to the cocktail treatment. The data for the markers HLA-D, CD83, CCR7, CD86, CD80, IL-12p70 and IL-23 are shown as fold induction of the cocktail treated cells compared to the respective immature DCs. The miRNA data are shown as log2 transformed data (corresponding to −ΔΔCt values). Statistical evaluation of the two groups showed that the levels of CCR7 (p<0.004) and IL-23 (p<0.019) were significantly different between the two groups (two sided t-test, unpaired). The other markers could not be shown to be significantly different between the two groups in this experiment. The miRNA levels were evaluated using a one class t-test, with the hypothesis that the miRNA levels are significantly different from 0. The good responder group showed significantly increased miRNA levels of hsa-miR-155, hsa-miR-146a, hsa-miR-125a-5p and hsa-miR-29a. The poor responder group showed significantly increased miRNA levels of hsa-miR-155, hsa-miR-146a.

The average marker level for each group was determined, and these data sets analysed using a two sided, unpaired t-test. Statistical evaluation showed difference between the levels of CCR7 and IL-23 levels between the two groups with p<0.004 and p<0.019 respectively (Table 13). Four miRNAs in particular were shown to be significantly induced in the cocktail matured DCs with good response (hsa-miR-155, hsa-miR-146a, hsa-miR-125a-5p and hsa-miR-29a) with a significance level at (p<0.0006, p<0.0003 p<0.02 and p<0.02 respectively) (Table 13, upper right part). In the poor responder group, hsa-miR-155 and hsa-miR-146a were also significantly induced, although at lower level of significance (p<0.03 and p<0.002 respectively) (Table 13, lower right part). The induction of hsa-miR-155 in the good responder group was also significantly different from the poor responder group (p<0.003) (Table 13, right part).

A comparison between the miRNA levels observed in the good responders and the poor responder group showed an increased miRNA level of hsa-miR-155, hsa-miR-146a, hsa-miR-125a-5p and hsa-miR-29a in the good responder group. However, only the hsa-miR-155 level could be shown to be significantly different between the two groups (p<0.003) in the present example. Thus, these markers, and particularly hsa-miR-155, may be used to distinguish between immature and immunogenic dendritic cells.

A significant correlation between CCR7 expression and the induction of hsa-miR-155 was observed when the data sets were plotted in a scatter diagram (R²=0.6979, P<0.02).

These data show that the functional state of immunogenic DCs can be predicted not only at the level of maturation markers (HLA-D, CD80, CD83, CD86, CCR7, IL-12p70 and IL-23), and in particular levels of CCR7 expression and IL-23 secretion, but also at the levels of particularly the four miRNAs of SEQ ID nr. 4, 27, 7 and 26 (hsa-miR-155, hsa-miR-146a, hsa-miR-125a-5p and hsa-miR-29a, respectively). In particular SEQ ID 4 (hsa-miR-155) increase can distinguish good responders from poor responders, where the good responding group increased hsa-miR-155 with 6.6 fold compared to immature DCs, whereas the poor responding group only increased hsa-miR-155 by 1.4 fold compared to their corresponding immature DCs.

### Example 6

### The Use of hsa-miR-155, hsa-miR-146a and hsa-miR-378 to Distinguish Between Untreated Tolerogenic DCs from Tolerogenic DC that are Exposed to Maturation Stimuli

As described in the beginning of the example section, DCs that are exposed to maturation stimuli (such as the maturation cocktail used in previous examples, TNFα, IL-1β, IL-6 and PGE₂) differentiate into a mature, immunogenic phenotype. However, DCs that undergo differentiation in the presence of tolerance-inducing agent such as 1α,25-dihydroxyvitamin D₃ become resistant to such maturation stimuli, and give rise to DCs with distinct phenotypes (table 3 and table 4). To distinguish those miRNA markers that are correlated with immunogenic function of DCs from markers that are differentially regulated merely in response to the maturation cocktail, the expression of hsa-miR-155, hsa-miR-146a and hsa-miR-378 was compared between immature DC, mature DC, vitatmin D₃-treated DC without exposure to maturation stimuli, and vitamin D₃-treated DC exposed to maturation stimuli.

TABLE 14

| −ΔΔCₜ values | microRNA | | |
|---|---|---|---|
| DC phenotype | hsa-miR-155 | hsa-miR146a | hsa-miR-378 |
| M (n = 4) | 3.1 ± 1.2* | 1.77 ± 0.53** | 0.04 ± 1.06 |
| M/VD3 (n = 4) | 0.09 ± 0.64 | 1.08 ± 0.67** | 0.69 ± 0.56* |
| IM/VD3 (n = 5) | −0.33 ± 0.55 | 0.09 ± 0.78 | 0.88 ± 0.72** |

Table 14. Determination of the level of induction of hsa-miR-155, hsa-miR-146a and hsa-miR-378 in immunogenic DCs (M) and in two different tolerogenic phenotypes (M/VD3 corresponding to the tolerogenic phenotype employed in the initial microarray based microRNA screening, and IM/VD3, which unlike M/VD3 was not exposed to a cocktail comprising TNFα, IL-1β, IL-6 and PGE₂). Significant differential expression of miRNAs in the different DC-phenotypes compared to immature DC is indicated by asterisks: *(P<0.05) and **(P<0.01).

Hsa-miR-155 was significantly enhanced only in immunogenic DC, suggesting that hsa-miR-155 can be used as a marker for immunogenic DC. In contrast, significant upregulation of hsa-miR-146a was observed in both immunogenic DC (M) and vitamin D₃-treated tolerogenic DC that are exposed to the maturation cocktail (M/VD3), suggesting that the upregulation of hsa-miR-146a is not an indicative of immunogenic function of DCs, but only an indication that differentiation from immature DCs has occurred into either tolerogenic or immunogenic DC. Hsa-miR-378 was significantly up-regulated in DCs treated with VD3 (M/VD3 and IM/VD3), which indicate that it may be a marker of the tolerogenic phenotype.

1. A method for monitoring the functional state of dendritic cells, comprising:
comparing an expression profile of one or more micro RNAs (miRNAs) which comprises a sequence that has at least 70% identity to a nucleic acid sequence represented by one of SEQ ID NOs: 1 to 45 to an expression profile of a reference sequence from a standard population of dendritic cells;
analyzing differential regulation of the one or more miRNAs to determine the functional state of the dendritic cells.

2. The method according to claim 1, further comprising distinguishing between immature and immunogenic dendritic cells, wherein the sequence is represented by one of SEQ ID NOs: 1 to 36 and the micro RNA is used as markers to distinguish between immature and immunogenic dendritic cells.

3. The method according to claim 2, wherein the sequence is represented by one of SEQ ID NOs: 1, 4, 5, 7, 9, 18, 26, 27, 28, 29, 30 and 31.

4. The method according to claim 3, wherein the sequence is represented by one of SEQ ID NOs: 4, 7, 26 and 27.

5. The method according to claim 1, further comprising distinguishing between immature and tolergenic dendritic cells, wherein the sequence is represented by one of SEQ ID NOs: 26 to 45 and the micro RNA is used as markers to distinguish between immature and tolerogenic dendritic cells.

6. The method according to claim 5, wherein the sequence is represented by done of SEQ ID NOs: 26, 27, 32, 34, 37, 39, 41 and 42.

7. The method according to claim 6, wherein the sequence is represented by one of SEQ ID NOs: 27 and 41.

8. The method according to claim 1, further comprising distinguishing between immature dendritic cells and tolerogenic or immunogenic dendritic cells, wherein the sequence is represented by one of SEQ ID NOs: 26 to 36 and the micro RNA is used as markers to distinguish between immature dendritic cells and tolerogenic or immunogenic dendritic cells.

9. The method according to claim 1, wherein the sequence has at least 90% identity to the reference nucleic acid sequence.

10. The method according to claim 1, wherein at least two different micro RNAs are used.

11. The method according to claim 1, wherein at least three different micro RNAs are used.

12. The method according to claim 1, where the dendritic cell is of human origin.

13. The method according to claim 1, wherein the dendritic cells being monitored are in therapeutic compositions comprising dendritic cells.

14. The method according to claim 13, wherein the therapeutic composition is a vaccine.

15. The method according to claim 13, wherein the therapeutic composition is used therapeutically for adoptive transfer.

16. A method of producing a quality controlled composition comprising dendritic cells, by which the functional state of the dendritic cells in the composition is monitored, the method comprising:
a) extracting nucleic acids from the dendritic cells from the composition,
b) monitoring the expression profile of one or more micro RNAs comprising a sequence that has at least 70% identity to a nucleic acid sequence selected from the group consisting of SEQ IDs 1 to 45 extracted in step a), and
c) comparing the expression profile obtained in b) with a standard profile from immature dendritic cells.

17. The method according to claim 16, wherein the sequence is represented by one of SEQ ID NOs: 1 to 36.

18. The method according to claim 17, wherein the sequence is represented by to one of SEQ ID NOs: 1, 4, 5, 7, 9, 18, 26, 27, 28, 29, 30 and 31.

19. The method according to claim 18, wherein the sequence is represented by to one of SEQ ID NOs: 4, 7, 26 and 27.

20. The method according to claim 16, wherein the sequence is represented by one of SEQ ID NOs: 26 to 45.

21. The method according to claim 20, wherein the sequence is represented by one of SEQ ID NOs: 26, 27, 32, 34, 37, 39, 41 and 42.

22. The method according to claim 21, wherein the sequence is represented by one of SEQ ID NOs: 27 and 41.

23. The method according to claim 16, wherein the sequence is represented by one of SEQ ID NOs: 26 to 36.

24. A quality-controlled therapeutic composition obtainable by the method of claims 16.

25.-28. (canceled)

29. A method for in vitro screening for compounds having immunomodulatory effect, comprising:
a) providing a test population of immature dendritic cells;
b) producing, from the test population, a micro RNA expression profile of at least one micro RNA comprising a sequence that is essentially homologous to a sequence represented by one of SEQ ID NOs: 1 to 45;
c) contacting the population of dendritic cells with a test compound;
d) producing a micro RNA expression profile as in step b); and
e) comparing the expression profiles obtained in step b) and step d), where a significant difference in profile will be indicative of immunomodulatory effect of the test compound.

30. The method according to claim 29, wherein the micro RNAs comprise a sequence that is essentially homologous to a sequence represented by one of SEQ ID NOs: 1 to 36.

31. The method according to claim 29, wherein the micro RNAs comprise a sequence that is essentially homologous to a sequence represented by one of SEQ ID NOs: 26 to 45.

32. The method according to claim 29, wherein the micro RNAs comprise a sequence that is essentially homologous to a sequence represented by one of SEQ ID NOs: 26 to 36.


SEQUENCE LISTING (45 sequences)

SEQ ID NO: 1 - RNA, 21 nt, Homo sapiens
uucacaguggcuaaguuccgc

SEQ ID NO: 2 - RNA, 21 nt, Homo sapiens
aucacauugccagggauuacc

SEQ ID NO: 3 - RNA, 21 nt, Homo sapiens
uucacaguggcuaaguucugc

SEQ ID NO: 4 - RNA, 23 nt, Homo sapiens
uuaaugcuaaucgugauaggggu

SEQ ID NO: 5 - RNA, 20 nt, Homo sapiens
agagguauagggcaugggaa

SEQ ID NO: 6 - RNA, 21 nt, Homo sapiens
aucacauugccagggauuucc

SEQ ID NO: 7 - RNA, 24 nt, Homo sapiens
ucccugagacccuuuaaccuguga

SEQ ID NO: 8 - RNA, 23 nt, Homo sapiens
cacucagccuugagggcacuuuc

SEQ ID NO: 9 - RNA, 21 nt, Homo sapiens
cuuuuugcggucugggcuugc

SEQ ID NO: 10 - RNA, 23 nt, Homo sapiens
uuucaagccagggggcguuuuuc

SEQ ID NO: 11 - RNA, 22 nt, Homo sapiens
aagcugccaguugaagaacugu

SEQ ID NO: 12 - RNA, 21 nt, Homo sapiens
cuccagagggaugcacuuucu

SEQ ID NO: 13 - RNA, 23 nt, Homo sapiens
aaugacacgaucacucccguuga

SEQ ID NO: 14 - RNA, 25 nt, Homo sapiens
agggaucgcgggcggguggcggccu

SEQ ID NO: 15 - RNA, 23 nt, Homo sapiens
gacacgggcgacagcugcggccc

SEQ ID NO: 16 - RNA, 22 nt, Homo sapiens
ugggucuuugcgggcgagauga

SEQ ID NO: 17 - RNA, 20 nt, Homo sapiens
acucaaacugugggggcacu

SEQ ID NO: 18 - RNA, 21 nt, Homo sapiens
caacaccagucgaugggcugu

SEQ ID NO: 19 - RNA, 22 nt, Homo sapiens
uggcucaguucagcaggaacag

SEQ ID NO: 20 - RNA, 22 nt, Homo sapiens
cggcggcggcggcggcggcugu

SEQ ID NO: 21 - RNA, 22 nt, Homo sapiens
uagcagcacguaaauauuggcg

SEQ ID NO: 22 - RNA, 22 nt, Homo sapiens
acucaaaaugggggcgcuuucc

SEQ ID NO: 23 - RNA, 20 nt, Homo sapiens
guccgcucggcgguggccca

SEQ ID NO: 24 - RNA, 22 nt, Homo sapiens
aaugcacccgggcaaggauucu

SEQ ID NO: 25 - RNA, 22 nt, Homo sapiens
aauguguagcaaaagacagaau

SEQ ID NO: 26 - RNA, 22 nt, Homo sapiens
uagcaccaucugaaaucgguua

SEQ ID NO: 27 - RNA, 22 nt, Homo sapiens
ugagaacugaauuccauggguu

SEQ ID NO: 28 - RNA, 23 nt, Homo sapiens
aggaagcccuggaggggcuggag

SEQ ID NO: 29 - RNA, 23 nt, Homo sapiens
aucccuugcaggggcuguugggu

SEQ ID NO: 30 - RNA, 22 nt, Homo sapiens
aggggcuggcuuuccucugguc

SEQ ID NO: 31 - RNA, 22 nt, Homo sapiens
ugcggggcuagggcuaacagca

SEQ ID NO: 32 - RNA, 22 nt, Homo sapiens
aacuggccuacaaagucccagu

SEQ ID NO: 33 - RNA, 22 nt, Homo sapiens
gcggcggcggcggaggcugcug

SEQ ID NO: 34 - RNA, 22 nt, Homo sapiens
ugagguaguaguuugugcuguu

SEQ ID NO: 35 - RNA, 23 nt, Homo sapiens
aggaccugcgggacaagauucuu

SEQ ID NO: 36 - RNA, 23 nt, Homo sapiens
guuugcacgggugggccuugucu

SEQ ID NO: 37 - RNA, 22 nt, Homo sapiens
uguaaacauccuacacucagcu

SEQ ID NO: 38 - RNA, 22 nt, Homo sapiens
uucaaguaauccaggauaggcu

SEQ ID NO: 39 - RNA, 23 nt, Homo sapiens
uccaguaccacgugucagggcca

SEQ ID NO: 40 - RNA, 22 nt, Homo sapiens
uguaaacauccucgacuggaag

SEQ ID NO: 41 - RNA, 21 nt, Homo sapiens
acuggacuuggagucagaagg

SEQ ID NO: 42 - RNA, 19 nt, Homo sapiens
aaaagcugaguugagaggg

SEQ ID NO: 43 - RNA, 22 nt, Homo sapiens
uggagagaaaggcaguuccuga

SEQ ID NO: 44 - RNA, 22 nt, Homo sapiens
ugagguaguagguugugugguu

SEQ ID NO: 45 - RNA, 22 nt, Homo sapiens
cugggagguggauguuuacuuc